(19) United States
(12) Patent Application Publication
(10) Pub. No.: US 2019/0223995 A1
(43) Pub. Date: Jul. 25, 2019

(54) DEVICE FOR DELIVERY AND RETRIEVAL OF PROTECTION FILTERS

(71) Applicant: Petrus A. BESSELINK, Enschede (NL)

(72) Inventor: Petrus A. BESSELINK, Enschede (NL)

(21) Appl. No.: 16/336,167

(22) PCT Filed: Sep. 25, 2017
(86) PCT No.: PCT/IB2017/001344

§ 371 Date: Mar. 25, 2019

Related U.S. Application Data
(60) Provisional application No. 62/458,646, filed on Feb. 14, 2017.
(60) Provisional application No. 62/425,762, filed on Nov. 23, 2016.
(60) Provisional application No. 62/399,623, filed on Sep. 26, 2016.

(51) Int. Cl. A61F 2/01

(52) U.S. Cl. CPC A61F 2/013; A61F 2002/9528; A61F 2002/011

(57) ABSTRACT

A device and method for the retrieval of endoluminal filters. In one form, a filter is a reverse-type device placed on the distal side of the operator such that the filter is placed in a direction where the blood flows towards the operator. This is particularly helpful when a filter is placed upstream such that it is moved in a direction against the blood flow. This permits closure of the filter mouth to initiate on the side where the blood enters to promote safe entrapment of emboli and other particles before the filter is completely collapsed and retrieved from the body. Such a reverse-type sheath can also be used in combination with other procedures. For example, it can be integrated with the same catheter device that is also used for angioplasty, ablation, stenting or valve replacement.

This application claims the benefit of U.S. Provisional Application Ser. No. 62/399,623, filed Sep. 26, 2016, U.S. Provisional Application Ser. No. 62/425,762, filed Nov. 23, 2016 and U.S. Provisional Application Ser. No. 62/458,646, filed Feb. 14, 2017.

### BACKGROUND

Filters for capturing particles from the blood stream are used in many interventional procedures and embodiments. Physicians say the key factors in choosing embolic protection filters are their ability to maintain perfusion, be effective in capturing emboli, a low profile, deliverability, retractibility and the ability to recover all the collected debris. Particles that have dimensions above a given critical size have to be trapped in order to prevent serious damage downstream. They can cause acute problems in organs like heart, brain, lungs, kidneys and others. Most filters are placed by means of delivery from a catheter sheath. Some filters stay in the body forever, like some vena cava filters, but in many cases they are only for temporary use and have to be retrieved. This is done by means of a retrieval sheath that is advanced over a guide wire that is attached to the proximal side of the filter. (In this description proximal means the side where the operator stands).

Therefore retrieval is easy in such cases, because the filter mouth, where the particles enter the filter, is also located at the proximal side of the operator. However, until now there was no solution for closing a filter mouth on the distal side of the operator, when filters are placed in a direction where the blood flows towards the operator. The present disclosure brings a solution for placing filters in these cases.

Filters may be used alone, in combination with balloon angioplasty or stenting or in combination with placement or replacement of a valve in a vein, artery or in the heart. Filter devices could protect patients' brains during not only trans arterial valve insertion, but also surgical aortic valve replacement, atrial fibrillation ablation and other left-heart procedures. Examples of other procedures that may cause the release of particles are the use of scoring balloons or lithotripsy balloons.

During a trans arterial valve insertion (TAVI) procedure balloon angioplasty of the native valve is followed by placement of a new valve. Normally a delivery sheath is withdrawn from the self-expanding stented valve in order to allow the full deployment of the valve. Alternatively a balloon expandable valve is placed, by inflating the balloon with the stented valve located on its outer surface. Such procedures should be done in combination with the use of a protection filter, in order to ensure entrapment of any emboli and other particles, dislodged during angioplasty and/or stented valve delivery. It is important that the side of the filter where the blood flow enters is closed first, before it is retrieved, in order to prevent squeezing out of particles upon reducing the outer dimensions of the filter, when it enters the retrieval sheath. However, in most cases the catheters that hold the balloon and/or valve have their distal end directed towards the blood flow direction. This makes it difficult to place a filter on a location more proximal, as seen from the operator's view. Several companies have developed additional catheters with a filter, but they do not have a solution for the closure of the distal entrance mouth of the filter before it is collapsed and retrieved.

Examples of existing embolic filters for TAVI operations are described in a website Dicardiology.com, specifically the Claret Medical Sentinel system, the Keystone Heart Triguard device, the Edwards Lifesciences Embrella deflector and the Emboline Inc. Prosheath embolic protection system.

Claret's Sentinel system is composed of a double embolic filter net, one for each carotid artery. The system combines a guidewire, support catheter and the two embolic nets into one catheter. Using a 6 French radial artery access point, the catheter is passed through the brachiocephalic artery into the aortic arch. There the tip is manipulated to make an extreme bend into the neighboring left common carotid artery and the first net is deployed just past the ostium inside the vessel. The second net is then deployed inside the brachiocephalic artery. These two filters only provide embolic protection for the brain, but do not stop debris that flows deeper into the descending aorta. One of the filters is closed first at the apex, so there is a risk of squeezing out debris upon retrieval.

Keystone Heart's Triguard device consists of a self-expanding nitinol frame covered in a mesh material to deflect emboli from traveling up any of the three vessels at the top of the aortic arch. It is deployed via a catheter introduced from the femoral artery. As the device is unsheathed, a nitinol stabilizing wire is expanded into the brachiocephalic artery to properly orientate the screen. A second stabilizing wire acts as a foot on the base of the device that touches the bottom of the aortic arch to help push the device upward to form a better seal against the top of the arch. After the procedure it is pulled back into the delivery catheter for removal. The net does not capture emboli, but does deflect it from the carotid vessels.

A third device discussed was the Edwards Lifesciences Embrella embolic deflector, which received European CE mark in 2010. The device is placed in the aorta through a sheath inserted in the right brachial or right radial artery. Its porous membrane allows blood flow to the brain while simultaneously deflecting embolic material. The device uses two self-expanding frames covered in a filter mesh, attached to a central catheter from which it is deployed and retrieved. This device is a deflector, similar to the Triguard device, and does not remove emboli from the body upon retrieval.

A fourth device, the Prosheath embolic protection system, deploys a large self-expanding stent structure with a filter mesh covering along the length of the aortic arch into the descending aorta. At the base of the device where it is attached to its deployment catheter, it uses a reversed funnel shaped filter to capture all emboli released during the procedure. It is capable of capturing all emboli released during TAVI procedures, because it deflects the emboli from the side arteries in the aortic arch into a location downstream into the aorta, where it is captured and retrieved in the filter part. However, in this device the filter is retrieved in a sheath from the proximal side, so the filter entrance is closed at the very end of the retrieval, which again may cause squeezing out of the captured particles.

All four devices are based upon an additional catheter holding the protection device. A separate catheter system is first placed before the procedure of angioplasty, stenting, placing a valve or whatever treatment is started. This costs extra equipment, operation time and it takes more place in the aorta. Moreover, there is always the risk of mechanical interaction between the protection device and the catheter that is inserted to do the TAVI procedure. Such mechanical interaction may lead to dislodgement of captured particles from the protection device or even an entanglement between the two devices which can cause huge problems.

There is still a need for an embolic protection system that is integrated with the treatment catheter, placed in the artery between the treatment site and the proximal operator and which can be closed and retrieved safely without the risk of squeezing out debris. Such a device can be used in many upstream procedures.

For downstream embolic protection in the carotid artery there is the Paladin device, having at its distal end a filter, integrated with a proximal angioplasty balloon on the same catheter, where the filter mouth is proximal and can be closed safely in such a downstream application. However, because of its construction this device cannot be used upstream and as of today there are still no devices that have a solution for closing the distal filter mouth.

Other filters for downstream use may include two filters on a single catheter such as that described in U.S. Pat. No. 6,485,502 by Don Michael and Besselink, in which several types of Nitinol frames are covered with a perforated polymer membrane, as well as one with a braided mesh with a specific pore size. In PCT Published Application WO 2004/026175 Besselink describes the use of fibers of high strength materials, embedded in polymer membranes to act as a reinforcement to enable the use of thinner membranes and prevent rupture and/or detachment from an expandable frame. These filters are also used as distal protection devices.

### SUMMARY

In one embodiment of the present disclosure, an embolic filtering system used to capture embolic debris in a body vessel with the blood flowing from the distal end towards the proximal end of the system is disclosed. The system includes an expandable and collapsible filter assembly with an expandable frame that can be moved between an expanded position and an unexpanded position, where the filter is disposed on a first tubular filter carrying catheter. A flexible filtering element is attached to and movable with the frame. The flow of unfiltered blood may be made to enter the filtering assembly at a first distal entrance side, while the filtered blood leaves the filter assembly at a second proximal exit side. The first filter carrying catheter has a proximal end outside the body and a distal section enclosed by the filter assembly with at least the distal end of the filter assembly being attached. A second tubular catheter is mounted coaxially with the first catheter and is movable in an axial direction relative to the first catheter. A reverse retrieval sheath is connected to the distal end of the second catheter, the sheath having a distal end portion and a proximal end, the distal end portion being connected to the second catheter and the proximal end portion of the sheath having an opening, adapted to maintaining the filter assembly in the unexpanded position, deliver the expandable filter assembly for release and being axially movable to collapse and retrieve the filter assembly, starting the collapse at the distal entrance of the filter; and a way for an operator to manipulate through attachment to the proximal ends of the first and second catheter in such a way to vary the relative axial position of both catheters.

In another embodiment of the present disclosure, a method of using an embolic filtering system is disclosed.

### BRIEF DESCRIPTION OF THE DRAWINGS

FIG. 1 shows a device with a control wire, a main catheter with a reverse sheath connected thereto and a set of retrieval wires.

FIGS. 2a and 2b show two different embodiments for the perforated connection ring between main catheter and reverse sheath.

FIG. 3 shows the same main catheter plus retrieval catheter as in FIG. 1, but now with an elongated sheath at the distal end.

FIGS. 4a-4d show different views of the device of FIG. 1.

FIG. 5 gives an example of a filter with an oval loop frame and a single retrieval wire, built on a catheter with a reverse sheath according to the disclosure.

FIGS. 6a-6f give several steps and details showing the delivery and retrieval of a single filter.

FIGS. 7a-7d show an embodiment with a filter mounted on a single separate filter holder tube.

FIGS. 8a-8e show another embodiment with a filter mounted on two separate tubes, which can be moved independently in axial direction, combined with an inserted balloon catheter.

FIGS. 9a-9e shows a version of the device of FIGS. 7a-7d, combined with a balloon catheter with a reverse sheath partly located inside the balloon section in order to enlarge the potential landing zone for the filter.

FIGS. 10a-10e show another type of device, where a self-expanding stent is delivered from the distal end of a delivery sheath, under protection of a filter with a reverse sheath on the same catheter.

FIG. 11 shows a schematic view of the aorta arch with two filter frames, holding a long cylindrical perforated membrane in between and a conical membrane at the proximal end.

FIGS. 12a-12d shows another embodiment and some details with the main catheter and reverse sheath being non-concentric.

FIGS. 13a-13c show another embodiment of a self-expanding filter that is collapsed without the use of a sheath.

FIGS. 14a-14c show another embodiment of a self-collapsing filter, without the use of a sheath, that is expanded with the help of a bias spring.

### DETAILED DESCRIPTION

In procedures where a filter is placed upstream, with its entrance for blood holding particles distally from the operator's position, the principle of a reverse sheath can be used. Closure of the filter mouth will then start on the side where the blood enters and this ensures safe entrapment of emboli and other particles before the filter is completely collapsed and retrieved from the body. The reverse sheath is mounted near the distal end of a main catheter, and its opening for retrieval of the filter is located more proximal. Retrieval is achieved by pulling the main catheter with the reverse sheath in proximal direction over the filter frame, while holding the filter in its axial position by means of one or more retrieval wires that are connected to a central control element that is held still upon collapsing the filter. Alternatively the filter is held still in its axial position by a separate filter holder tube, which is placed over the main catheter that holds the sheath that is used for delivery and retrieval of the filter.

It will be understood that the reverse sheath can also be used in combination with other procedures. In the present description, examples are given for the use of such a reverse sheath, integrated with the same catheter device that is also used for angioplasty, stenting or valve replacement, but it can also be used separately besides these devices. For all kinds of upstream treatments resulting in the release of particles, valve tissue, arterial debris, calcification, thrombus or foreign material the filter with a distal blood entrance, eventually retrieved by means of a reverse sheath can provide protection. In another embodiment of a filter with a distal entrance, used in an upstream procedure, but where the room for using a reverse sheath is too small, an example is given of a filter that is expanded or collapsed by means of two concentric tubes, which are independently movable in axial direction. These concentric tubes can slide back and forth over the surface of a standard device for treatment in the patient's vascular system. The relative movement of both tubes cause a length change of the filter struts, resulting in a change of the outer diameter of the filter frame.

The integration of the catheter for any treatment at the distal end in combination with an embolic filter on the same catheter shaft safes precious operation time, reduces costs as compared to separate devices and avoids the risk of undesirable mechanical interaction, including entanglement between separate systems.

Other embodiments include filters with or without a retrieval sheath, either placed concentric or non-concentric over the catheter for upstream treatments in the patient's body.

In the present description, the term “proximal” means the side where the operator stands. Filters according to the disclosure are placed upstream, with the blood flow running from distal to proximal, as seen by the operator. The term “reverse sheath” is used to define a sheath that has its open side directed towards the operator, in contrary to conventional sheaths where the opening is directed distally. The reverse sheath needs to be connected to the main catheter in order to enable the operator to move the sheath back and forth over a central control wire. In a first embodiment the place of the connection between the main catheter and reverse sheath is realized with a perforated connection ring. The perforation through the wall of the ring is needed to guide at least one retrieval wire through, which is connected to an expandable filter frame on the proximal side. The distal end of the retrieval wire is either directly connected to the central control wire or via a control ring, attached on the central control wire at a location distally from the perforated ring. Axial sliding of the main catheter relative to the central control wire causes a movement of the retrieval wires in and out of the reverse sheath, thus enabling the operator to pull the filter frame into this sheath.

Examples are disclosed for use of a reverse sheath in a single filter, a filter in combination with a balloon and for a valve replacement under filter protection. Other examples show a sheathless filter for upstream protection, also in combination with a balloon catheter.

However, for all kinds of upstream treatments resulting in the release of particles, valve tissue, arterial debris, calcification, thrombus or foreign material the filter with reverse sheath can provide protection. Such treatments include intravascular imaging, angioplasty, ablation, use of drug-eluting balloons, scoring balloons, lithoplasty, ultrasonic treatments, placement of closing systems like Left Atrial Appendage Occluders and many more.

In one form, a central lumen may be made to extend all the way between the proximal and distal end of the system. In one form, a diagnostic or interventional treatment catheter may extend through the lumen. Likewise, in one form, the distal end of the treatment catheter may be placed at a location distally of the landing zone where the filter is placed. Moreover, the treatment may include procedures such as intravascular imaging, angioplasty, dissection, ablation, use of drug-eluting balloons, scoring balloons, laser cutting, drilling, reaming, dilating, lithoplasty, ultrasonic treatments, placement of stents, valves, stented valves, occluders, permanent filters and closure devices.

Single Filter

A first example present in this disclosure is based on a filter, used without additional treatment devices. A reverse sheath for retrieval of the filter is mounted on a perforated connection ring near the distal end of a main catheter, with its retrieval opening directed to the proximal end of this catheter. Closure of the filter mouth, where the blood enters the filter, is achieved by pulling the retrieval sheath in proximal direction over the filter. By doing this, the particles that are collected in the filter, cannot be squeezed out because the filter entrance is entirely closed now. During closing the filter must be held in place while the sheath is moved over the filter surface. Retrieval wires that are connected to the distal end of the filter frame and to a control ring, mounted on a central control wire hold the filter in place. These retrieval wires, running between the outer surface of the main catheter and the inner surface of the reverse sheath, leave the distal end of the main catheter through the perforations in the perforated connection ring and are directly attached to a control ring on the central control wire. Latter attachment may be by means of shape fit, crimping, welding, soldering, glue or any other conventional technique.

The distal section of the control wire may have a floppy or steerable end, dependent on specific applications. Further the control wire in all following examples may be solid or tubular, for example for insertion of a guide wire or injecting contrast fluid through its inner lumen.

When the control wire is held in place and the reverse sheath is moved to proximal, the retrieval wires are stretched and the tensile force in these wires pulls the filter into the gap between main catheter and reverse sheath. Any type of filter may be used in combination with a reverse sheath catheter according to the present disclosure. This includes nitinol frames with a perforated polymer membrane or vena cava filters entirely made of metal. Retrieval of the latter filter, where the apex of the filter is pulled into the reverse sheath would occur with the blood flow in distal direction, in contrary with the other examples.

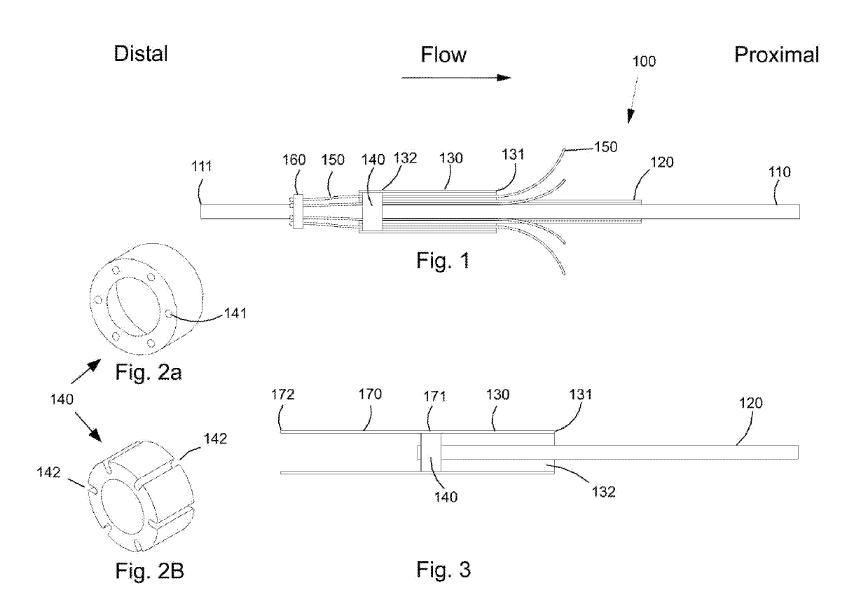

FIG. 1 shows a device 100 with a central control wire 110, a main catheter 120 with a reverse sheath 130 connected thereto via perforated connection ring 140 and a set of six retrieval wires 150, running through openings 141 in ring 140. Distally the retrieval wires are connected directly to the surface of the control wire 110, or to a ring 160, which is mounted on that control wire. Movement of the main catheter in proximal direction relative to a fixed position of the control wire will move the control wires through the gap between the inner surface of the reverse sheath and the outer surface of the main catheter. On the proximal side the retrieval wires are attached to an expandable filter frame, which is not shown here. Retrieval wires can be made of any material, including metal, shape memory material like nitinol with linear elasticity or superelastic behavior, polymer, high strength polymer (for example Kevlar or Dyneema fibers), carbon, ceramic fibers or others. The present inventor hereby refers to embodiments of filters, as described in PCT Published Application WO 2004/026175 the disclosure of which is incorporated by reference. The wires can also be elongated sections of the filter frame itself. In the latter case they may be part of a basket, which support an additional perforated polymer membrane that is attached to the basket frame. However, the basket itself can also act as a filter, depending on the mesh size and the dimensions of the particles, which have to be captured. The basket may also be made of a woven wire mesh. In the following drawings the basket is represented as an expandable nitinol frame, laser cut from tubing and shape set in order to become self-expanding properties. The retrieval wires are flexible and they are attached to connection points on the distal side of the frame. On the proximal side of the frame a perforated polymer membrane with fiber reinforcement is attached. Any other filter can be modified for use in a similar way in combination with the reverse sheath, and its use should be considered as been included in the embodiments of the disclosure.

The connection of sheath 130 to catheter 120 via ring 140 can be made by any known technique. In a specific example ring 140 may be one integrated part of the distal end of the main catheter. After placement of the retrieval wires the reverse sheath may be positioned on ring 140 and glued or crimped to its outer surface.

FIGS. 2a and 2b show two different embodiments for the perforated connection ring 140 between main catheter 120 and reverse sheath 130. In FIG. 2a the ring has holes 141, where the retrieval wires 150 can be put through, but it may be cumbersome to do this in a device where the sizes are extremely small. For example, if the outer diameter of the reverse sheath is as small as only 1 mm, it is clear that the retrieval wires may only have a diameter of 100 micron or less, which makes them difficult to handle.

FIG. 2b shows an embodiment for a ring 140, where the retrieval wires can easily be put into the half holes or slits 142 in the ring in radial direction. This is possible because the holes meet the outer surface of the ring. After placing the retrieval wires the reverse sheath is mounted on the 140, as mentioned before. Other embodiments of a ring 140 are possible as well. It may be a short section of the distal end main catheter with an increased wall thickness and one or more radial slits, like the ones in the ring of FIG. 2b, for example.

FIG. 3 shows the same main catheter 120 plus retrieval catheter 130 as in FIG. 1, but now with an elongated sheath 170 at the distal end. As described before the elongated sheath can be attached to ring 140 in a similar way. In FIG. 3 the control wire is not shown. It will run through the centre of the main catheter in a manner similar to that depicted in FIG. 1.

The elongated distal sheath 170 has a proximal end 171 and a distal end 172 and it can be used for delivery of other devices under the protection of the deployed filter downstream. The elongated sheath 170 can hold a stent or a stented valve, for example. Withdrawal of the sheath over the control wire 110 will push the stent out of distal end 172, while the reverse sheath moves simultaneously towards the filter mouth, which it will engage at its proximal end 131 first. (The filter itself is not shown here).

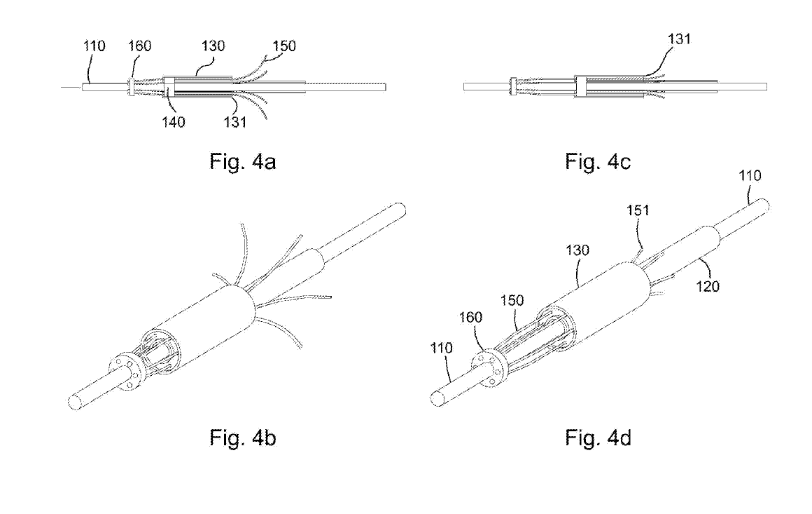

FIGS. 4a-4d show different views of the device of FIG. 1. FIGS. 4a and 4b give a side view and perspective view, respectively, with the control ring 160 in its position close to the ring 140. This would be the position where the filter is in its deployed state. The proximal ends of the retrieval wires extend widely from the proximal end of the reverse sheath.

FIGS. 4c and 4d give a side view and perspective view, respectively, of the catheter in its state where the reverse sheath is moved proximally over the central control wire. The control ring 160 pulls the retrieval wires 150 into the reverse sheath and this will result in the collapse of the filter frame and finally the entire frame can enter the gap between the inner surface of the sheath 130 and the outer surface of main catheter 120.

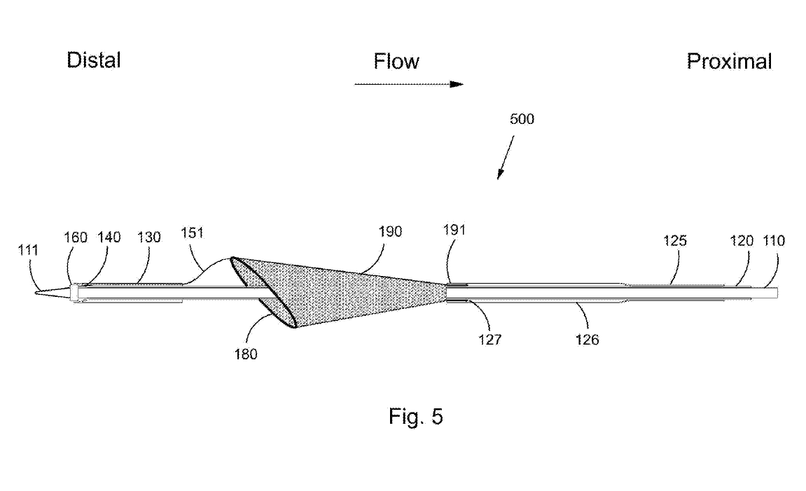

FIG. 5 gives an example of a filter 500 with an oval loop frame 180 which is attached at the distal end of a perforated membrane 190. The filter 500 is built on a main catheter 120 with a reverse sheath 130, mounted at its distal end with a perforated ring 140 according to the disclosure. Control wire 110 runs through the central lumen inside the main catheter and holds an atraumatic soft tip 111 and a control ring 160 at its distal end. Frame 180 has a single retrieval wire 151, connected to a ring 160 near the distal tip 111 of control wire 110. The parts are similar to the ones described in FIG. 1, but now there is only one retrieval wire. A separate delivery catheter 125 is located at the proximal side of the filter and only covers the membrane 190 in a filter surrounding section 126 with enlarged diameter. Delivery of the filter is achieved by pulling the delivery catheter 125 in proximal direction off the membrane, while the main catheter 120 is held still. Eventually the delivery catheter 125 may only be connected to the proximal end of the filter membrane 190 at control ring 191, which can move back and forth freely over the main catheter 120. The membrane is then uncovered during insertion and retrieval. The oval loop frame is stored in the reverse sheath 130 and upon delivery it is pulled out of this sheath by the movement of the membrane when delivery catheter 125 is pulled in proximal direction.

Retrieval of the filter is caused by holding the control wire 110 still and pulling the main catheter 120 into proximal direction.

This applies tension on the retrieval wire 151, finally causing deformation and collapse of the loop when it meets the proximal end of the reverse sheath. After the dimensions of the loop have changed enough, the moving sheath can enclose it completely. It may not be necessary to pull the entire filter membrane into the sheath, because closure of the loop is sufficient to prevent squeezing out of captured debris. The single retrieval wire 151 can be made of metal, like nitinol, but also may be made of a more flexible high strength polymer, which makes it possible to wrap it up in the gap between main catheter and retrieval sheath when the filter is loaded in the sheath before insertion into the patient. This can make the sheath shorter.

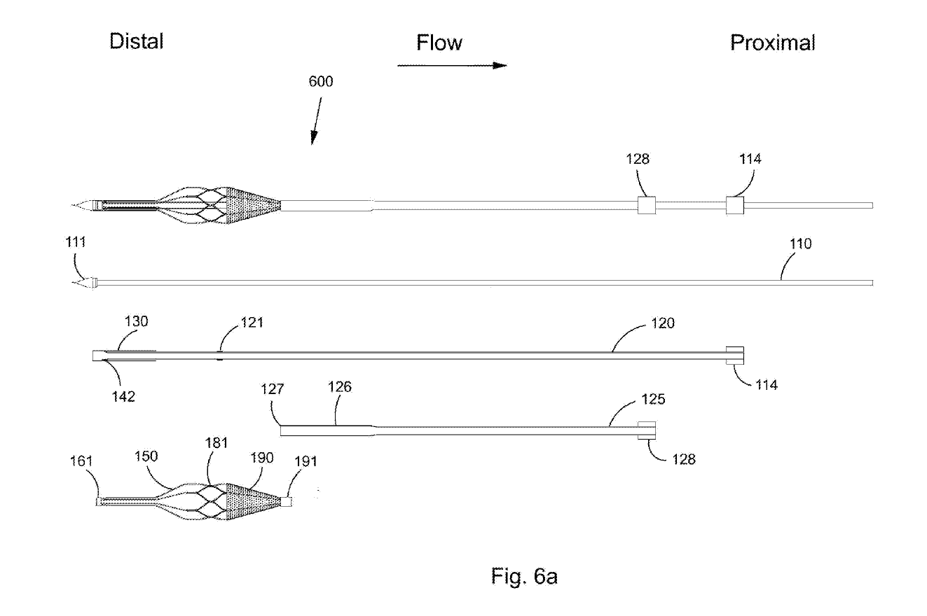

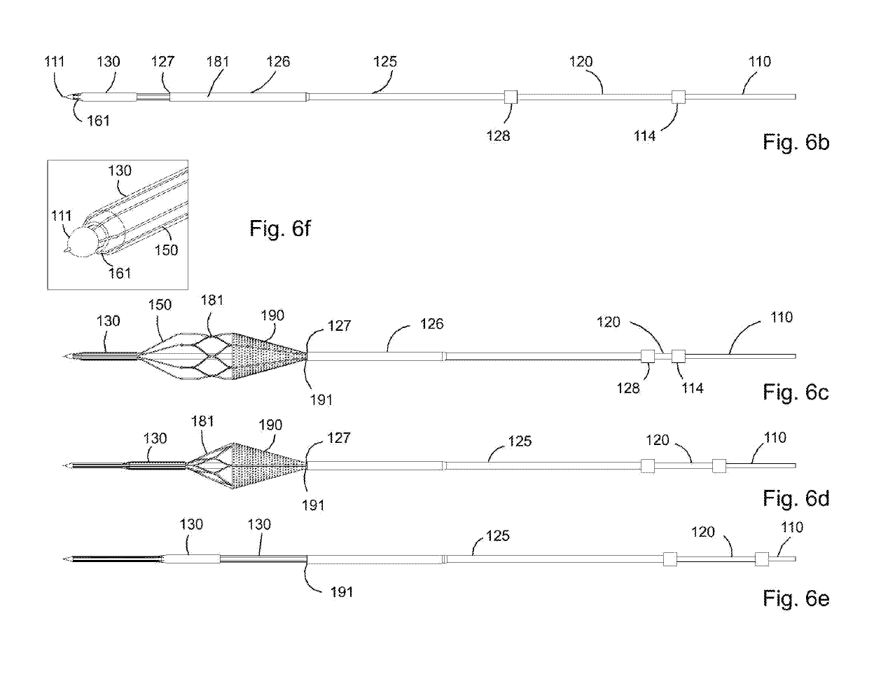

In the following FIGS. 6a-6f through 11, filters with a self-expandable frame made of a six-cell diamond structure with elongated legs on the distal end which are connected to retrieval wires are shown. These embodiments are shown just as an example, but it should be understood that other filter geometries can be used as well.

FIG. 6a shows an exploded view of the components of one embodiment of a single filter like described in FIG. 4. In FIG. 6a the separate parts are a shown in their relative position as given in FIG. 6c. There is a control wire 110 with a floppy distal tip 111. Eventually this wire is hollow and allows the insertion of the control wire over a separate guidewire (not shown). The filter itself has three sections. It has a self-expanding frame 181, six retrieval wires 150 connected thereto at its distal end and a perforated polymer membrane or wire mesh 190 attached to its proximal side. The distal ends of the retrieval wires are connected to control wire 110 with ring 161 near tip 111. The proximal end of filter 190 is connected to ring 191, which slides over main catheter 120. As with the embodiment depicted in FIG. 5, an additional delivery catheter 125 can cover the filter during insertion in wider section 126 and uncover it by pulling catheter 125 in proximal direction off the filter. Section 126 has a distal end 127 and during deployment this distal end 127 is moved proximally all the way until it is located near the membrane apex at ring 191.

During use the blood enters through the wide open distal frame entrance and is then filtered by membrane or mesh 190. FIGS. 6b-e show the parts of the catheter of FIG. 6a mounted together, shown in different stages during the procedure. All components are shown in their present relative axial positions.

FIG. 6b shows the embodiment of FIG. 6a, where almost the entire filter membrane is loaded in a separate second delivery sheath 125, which is located on the proximal side of the filter frame 181. A small stop ring 121, mounted on the main catheter surface, may be helpful to pull the filter membrane into sheath 125 upon loading. This can be done by placing the filter with its frame 181, membrane 190 and control ring 161 over the proximal end of the main catheter and slide it all the way until the control ring engages with the stop ring 121. Then wider section 126 of catheter 125 can be moved over the filter. Only the retrieval wires and distal control ring 161 are not located inside 126 and can be attached later. The position of the collapsed frame 181 is depicted, although it is covered by sheath 126. In addition, the filter frame itself is also loaded in the delivery sheath 126, but it is also possible to load the frame into sheath 130, while the remainder of the filter is loaded in the second sheath 126.

The filter is brought in place in collapsed state. At the proximal ends of main catheter 120 and sheath 125, outside the patient's body, manipulators or stops 114 and 128 are attached. These manipulators enable the operator to hold and move the separate parts in a reliable and smooth way. Any tool may be used for this manipulation, and the manipulators are only depicted schematically as small rectangular stops in the figures.

FIG. 6c shows the next step, where the filter is fully deployed by pulling it out of either or both sheaths 126 or 130, by moving 125 in proximal direction over the main catheter. Meanwhile the control wire and main catheter are kept still. As soon as the filter frame leaves its surrounding delivery sheath, it will expand and meet the artery wall. In the version of FIG. 6b the location where the filter is placed will be directly proximal of the proximal end of the reverse sheath 130. After expansion the filter frame should not be moved anymore along the artery wall in order to prevent spasms or damage.

In the shown examples of FIGS. 6a-6f, the distal end 127 of the delivery catheter 125 may have a slightly smaller inner diameter than the outer diameter of the control ring 191, so that it can exert a pull force to that control ring.

FIG. 6d gives the step of closing the frame, where reverse sheath 130 is moved over the filter frame 181 by pulling the main catheter 120 in proximal direction, while the control wire 110 is kept still. In this end position the filter mouth of the frame is closed gradually, while the volume of the still open conical membrane does not change. Finally, at the end of collapsing of the frame, the filter mouth is closed completely and the debris in the membrane is safely entrapped. In FIG. 6e the final situation is shown and the filter plus catheter can be removed from the patient together. Optionally, the distal section 126 of catheter 125, which is wider than the proximal end, may be pushed over the filter membrane, which holds the entrapped particles. It is used as a retrieval sheath for the membrane then, while 130 is the retrieval sheath for the filter frame, which had to be closed first.

Filter Holder Tube

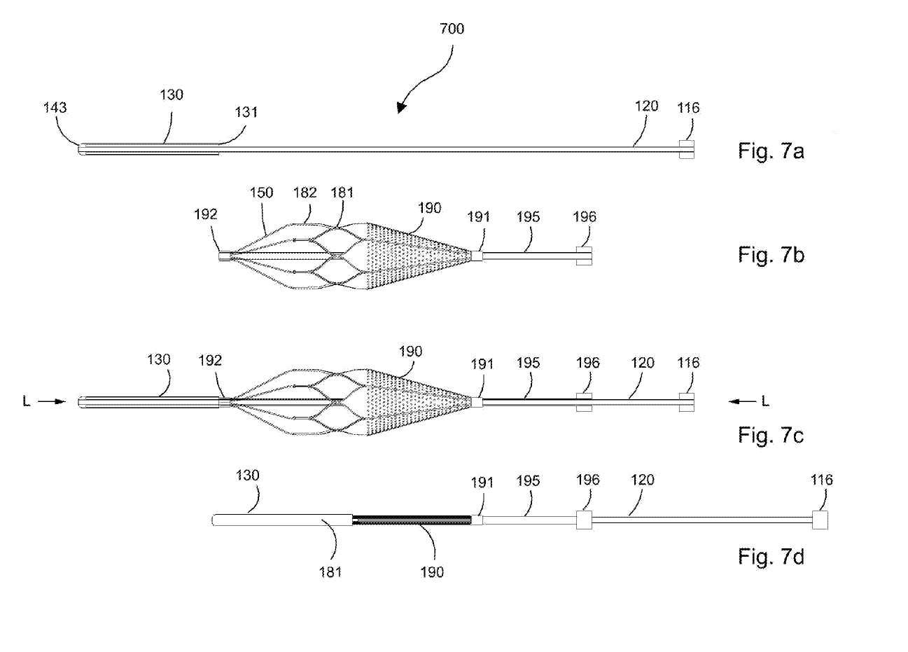

In the examples described above a control wire inside the main catheter is used to pull the filter into the sheath by means of retrieval wires. Another example of an embodiment of the disclosure is given as device 700 in FIGS. 7a-d. A tubular filter holder is mounted around a main catheter that holds the reverse sheath at its distal end. Such a combination of a main catheter with a filter holder tube can have a relatively large internal lumen and can be mounted on any existing device for intraluminal procedures, thus providing integrated coaxial protection.

FIG. 7a shows the tubular main catheter 120 and mounted thereto at its distal end a ring 143, holding a reverse sheath 130, having its opening 131 in proximal direction. The central lumen L of the main catheter, depicted in FIG. 7c, which runs through its entire length, allows the use of an additional guide wire, so that the main catheter can be guided into the patient's body. Lumen L can be made large enough to insert a catheter through it for example for a balloon angioplasty or a catheter for stent or valve delivery, as will be described as follows.

FIG. 7b shows a filter frame 181 with retrieval wires 150 connected to a distal ring 192, and a filter membrane 190 with an apex connected to a proximal ring 191. Both rings are mounted on a tubular filter holder 195, which can slide back and forth in its entirety over the main catheter 120. Such a filter holder tube may look like a larger version of so called “over the wire” or “rapid exchange” catheters that are used in combination with guidewires. The difference however is that the filter holder tube runs not over a guidewire, but over a catheter in some embodiments. Alternatively, a guidewire may be inserted first, and the filter holder tube is inserted over this guidewire and the filter is placed. Thereafter an additional device can be inserted over the guidewire and through the lumen of the filter holder tube, while the filter stays deployed. The filter holder tube has a long end at its proximal side and runs all the way to the manipulation tool of the operator (not shown). This tool may be connected to manipulator ring 196 at the proximal end of the filter holder tube. Both connection rings 192 and 191 can be made of a radio-opaque material in order to know the filter position exactly during the procedure. For the same purpose another radio-opaque ring can be connected near the entrance 131 on sheath 130. It may be clear that the mechanical properties of the filter holder tube depend on the desired function. Axial flexibility has to be combined with sufficiently low profile and strength for moving the filter in and out of the sheaths. A method to increase the axial flexibility, while maintaining enough stability would be to make the filter holder tube of thin walled braided tubing, provide it locally with a pattern of slots or even make it in the shape of a helical spring, for example.

The distance between the two rings 191 and 192 is sufficient to allow the filter frame to move easily in radial direction relative to the surface of the filter holder. Details associated with such filters are disclosed in patents EP1539031, JP4440787 and published US application 2006/015136 the details of which are incorporated by reference in their entirety. An important feature of the design is that the expanded filter frame ensures a perfect wall apposition, preventing any blood to pass around the filter mouth. Radial and tangential movements of the catheter in the bent aortic arch do not influence the shape and wall apposition of the filter frame, because the membrane and retrieval wires are extremely flexible. This eliminates most of potential mechanical interaction between filter and catheter. Normally the filter frame is made of laser cut Nitinol tubing with linear elasticity or superelasticity, but it will be appreciated that alternative materials may be used, and that all such variants are deemed to be within the scope of the present disclosure.

FIG. 7c gives the main catheter of FIG. 7a and the filter holder plus filter of FIG. 7b mounted together, in a position where the filter is in its deployed state. When the filter has to be collapsed and retrieved, the only thing that the operator has to do is holding the filter holder steady and pulling the main catheter into proximal direction until the reverse sheath has enclosed the filter frame. It is helpful when the distal end of the filter holder is slightly tapered with a nose cone at ring 192 to ensure that it slides easily into the entrance 131 of reverse sheath 130. If needed, the sheath may be long enough to enclose the membrane as well, but only enclosing the frame works as well. This is shown in FIG. 7d, where the reverse sheath 130 completely covers the collapsed filter frame 181 after the main catheter is pulled into proximal direction while the filter holder is kept still. Mounting the filter on a single filter holder tube has one disadvantage. Upon collapsing, the conical filter membrane, the frame itself and the retrieval wires all will get a larger axial length and therefore the membrane will get creased somehow.

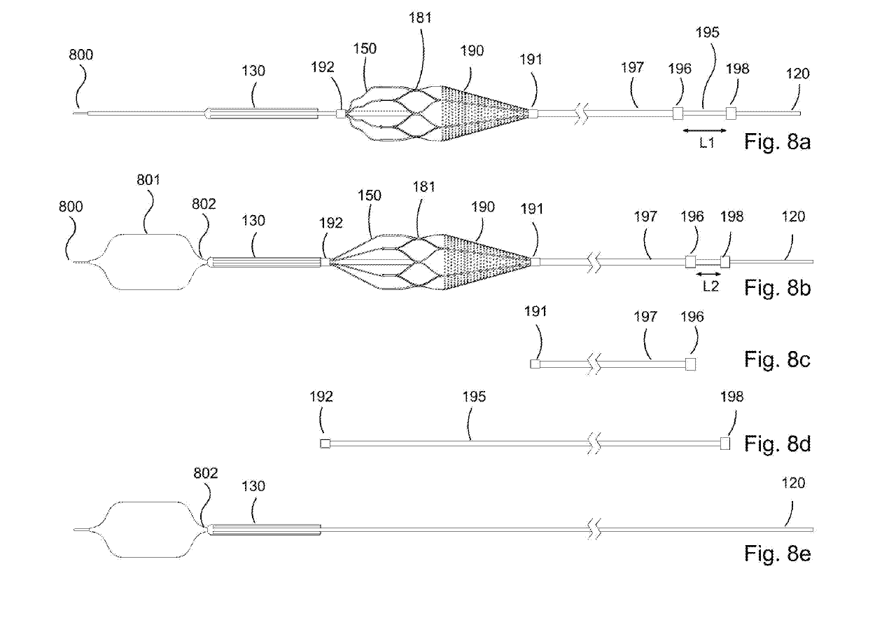

Therefore a slightly different design is showed in FIGS. 8a-e, where rings 192 and 191 are not mounted on the same filter holder tube. Ring 192 still connects the retrieval wires to the distal end of a smaller filter holder tube 195, but ring 191 connects the tip of the filter membrane 190 directly to an additional outer tube 197 that can move back and forth over the surface of the filter holder tube 195. Both tubes 195 and 197 have proximal manipulator rings 198 and 196, respectively. The operator can pull the two rings 198 and 196 close to each other to stretch the filter and make withdrawal into the sheath 130 easier and prevent creasing of the membrane. Both tubes 197 and 195 are then held still together, while the main catheter 120 is moved in proximal direction in order to pull the sheath 130 over the filter frame. However, when the filter is in its open state, he operator can increase the distance between the rings 196 and 198 in order to give the retrieval wires 150 some free play, as can be seen by comparing the length L1 and L2 between both rings in FIGS. 8a and 8b. This results in less mechanical interaction between the catheter and the filter frame in tangential and radial direction. In a strongly bent artery, like in the aortic arch, this ensures a better wall apposition of the filter frame, independent from the position and movements of the catheter. The extra tube 197 is also needed to pull the filter out of the sheath when it is placed.

The combination of main catheter and filter holder tube, eventually with the extra tube 197 can slide over a central guide wire, but also over a balloon catheter or a TAVI delivery catheter, for example. The balloon may be used for any procedure as described before. It may be a bare balloon, but it can also hold a balloon expandable stent, eventually in combination with a valve mounted to the stent frame (not shown here).

In FIGS. 8a-8e such a balloon catheter is shown, with a guidewire 800 and main catheter 120 carrying an inflated balloon 801 at its distal end. When the retrieval sheath is mounted directly on the balloon catheter, like shown at location 802, the relative axial position between balloon and sheath cannot be varied. However, with the sheath mounted on main catheter 120 and a separate balloon catheter running through main catheter 120, the axial position where the filter is deployed can be independent of the present location of the balloon or the stent delivery sheath, so the system is very flexible. The operator then can choose when and where he wants to deploy or retrieve the filter, while the procedure for angioplasty or valve delivery takes place as he wishes. It is even possible to reposition the filter by dragging it in axial direction while deployed, but this may cause damage or spasms in the artery wall. Therefor it is better to first collapse, then reposition and deploy the filter again, while the balloon or valve delivery device remains still exactly in its ideal location. The opposite is possible as well, when the filter stays in its position, while the balloon or valve delivery catheter are being repositioned.

Filtration can still continue after deflation of the balloon and when the desirable filtration time has expired, the main catheter 120 can be pulled in proximal direction while the filter is held still with catheter 197.

The principle of a filter holder tube may be used in combination with various filter types. One example would be the filter with a loop frame like the one shown in FIG. 5, mounted at the distal end of a filter holder tube. Delivery and retrieval would occur in a similar way as for FIGS. 7a-7d and 8a-8e.

Balloon with Internal Retrieval Sheath

When there is not enough axial space available to place the filter frame in a proper landing zone, the device can be made shorter by combining the balloon with the sheath. This can be done by placing at least a part of the sheath inside the balloon, so that the sheath and balloon act together as an inflatable sheath.

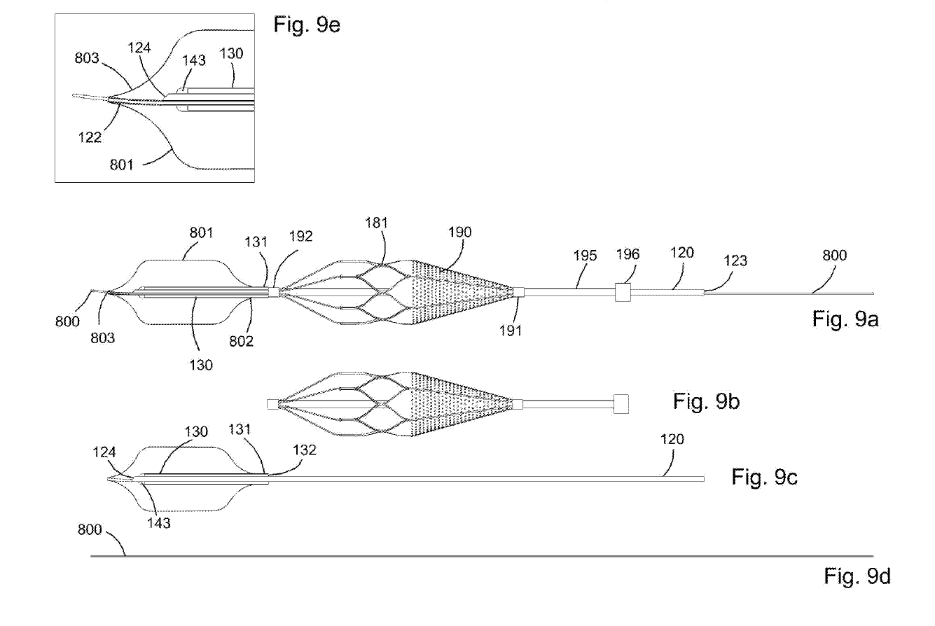

FIGS. 9a-e show a shorter version of the device of FIGS. 8a-e, with a filter holder tube, but now with the reverse sheath partly located inside the balloon section. In FIG. 9b the filter holder tube and filter are shown separately and in FIG. 9a they are assembled on the delivery and retrieval system.

In FIG. 9e an enlarged detail of the distal section of the device of FIGS. 9a and 9c is shown, with a relatively rigid sheath 130, which is enclosed inside the balloon 801. The inflation port 124 for realizing the balloon expansion and deflation is located close to the distal balloon tip 803, and the fluid flows through a separate lumen 123 in the main catheter 120, which also has an extra lumen 122 for a guidewire 800. For clarity lumen 122 for the guidewire is depicted as a very thin tube, but it may be clear that the geometry of the parts is only shown schematically. The guidewire is shown separately in FIG. 9d. The main catheter 120 holds the distal end of the balloon, while the proximal end 802 of the balloon is mounted on the outer surface 131 of the reverse sheath 130. More proximal, the entrance 132 of the reverse sheath is preferably located outside of the balloon area, in order to prevent eventual damage to this entrance by the high inward balloon pressure. The entrance tip at 131 can be made more rigid, for example by using a metallic marker band, eventually with a slight taper at the most proximal end to facilitate an easier swallowing of the filter. If the sheath inside the balloon section is rigid enough, it will stay cylindrical despite the high pressure. However, the total diameter of such a device, with the balloon material surrounding the sheath, may become too large for specific procedures.

Therefore, in another embodiment a more flexible sheath material inside the balloon section may be used. The sheath entrance 131 can still be more rigid, because it is outside the balloon. When the balloon is inflated, it will expand outside but it also pushes the enclosed sheath 130 against the outer surface of its underlying catheter 120, which is also used to pull the sheath over the filter after the balloon is deflated. When the material of the sheath is made elastically enough in radial direction, it can unfold or expand back to a similar diameter as the remainder of the sheath outside the balloon area, as soon as the balloon is deflated. By choosing the right compliance of the balloon and sheath material, eventually in combination with the use of reinforcement fibers, the behavior of this balloon/sheath combination can be optimized. In one example, the balloon material is elastic enough to maintain a circular cross section during the complete inflation/deflation process. Reinforcement fibers embedded in the balloon surface may limit the maximum balloon diameter to prevent burst. It may be clear that the sheath material must be able to withstand enough axial compression forces without buckling in order to be able to retrieve the filter.

When the deflated balloon/sheath is pulled into proximal direction in order to collapse the filter frame, the proximal opening of the sheath first encloses the filter mouth, and upon further axial withdrawal of the sheath also the flexible section of the sheath expands to its nominal size and takes up the remainder of the filter frame. With such a combined sheath/balloon a considerable gain is reached in the possible landing zone for a filter between the aortic valve and the brachio-cephalic artery. Other components in FIGS. 9a-9e are similar to the ones described in FIGS. 7a-d.

Optionally a balloon catheter may also be provided with a perfusion lumen (not shown), if it has to be used for a longer period, for example when drug delivery from the balloon surface is taking time. An example of a such a perfusion balloon is described in U.S. Pat. No. 6,776,771 by Van Moorleghem and Besselink the details of which are incorporated by reference in their entirety. Such devices may be combined with a reverse sheath as described in the present disclosure.

The perfusion lumen can be used as a reverse sheath as well, similar to the examples of FIGS. 9a-9e, where the rigid sheath holds the lumen open during inflation. In that case there needs to be an open connection to the distal tip of the balloon and the blood can flow into the distal end of the sheath lumen and leave it again at the proximal mouth of the reverse sheath. Catheter 121 is used for inflation/deflation of the balloon by means of at least one inflation port, located close to the distal end of the sheath/balloon in order to enable withdrawal of the filter frame into the sheath. This inflation port connects the fluid lumen of 121 with the space between the outer wall of the sheath and the inner wall of the balloon. Therefor the sheath combined the function of perfusion lumen with collapsing the filter frame for retrieval.

Placing an Integrated Self-Expanding Stent or TAVI-Valve with a Filter

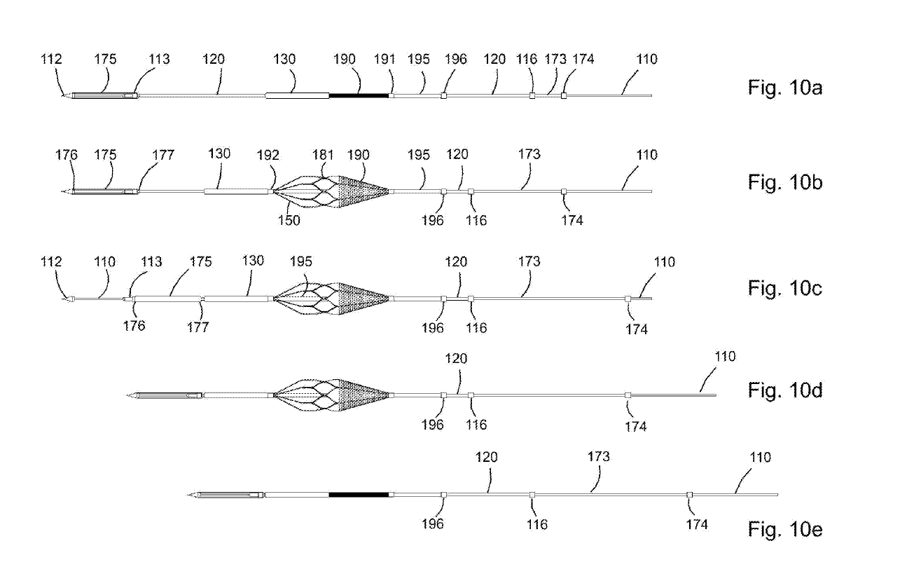

FIGS. 10a-e show another type of procedure, where a self-expanding stent is delivered from the distal end of a valve delivery sheath in a manner similar to that of the embodiment depicted in FIG. 3, again under protection of a filter on the same catheter. The stent can also be a stent frame, holding a valve.

In this example the filter has to be placed further away from the proximal end of the stent than from the balloon in FIGS. 8a-8e, because the delivery sheath needs room to be withdrawn from the stent entirely. This extra distance depends on the stent length and should be sufficient to have the filter still deployed while the stent is released completely. Again, it is important to place at least the filter frame between the location where stent and/or valve are placed and the first side artery. The perforated membrane may be located over the side artery, thus giving full protection there, but also in the main stream. The distance between a typical aorta valve and the first side artery is sufficient for placing the filter and still having room to withdraw the delivery sheath from a stented valve. The filter can then be collapsed later, as is shown in the FIGS. 10a-e.

The insertion state of the stent, valve and filter of the device is depicted in FIG. 10a. A control wire 110, with a distal tip 112 and a few centimeters more proximal a stop 113 mounted on the same wire. Over wire 110 a tubular valve delivery catheter 173, with a valve delivery sheath 175 at the distal end and a manipulator or stop 174 at the proximal end. In one form, an entire catheter may be configured to extend entirely over an insertion guidewire (not shown). Inside the gap between tip 112 and stop 113 the control wire is surrounded by a self-expandable stent frame with an aortic valve attached thereto (not shown). The frame and valve are held in their collapsed state by the surrounding valve delivery sheath 175, between its distal end 176 and proximal end 177.

A separate main catheter 120 with a reverse sheath 130 runs over valve delivery catheter 173. Main catheter 120 carries a filter holder tube 195 and a filter with frame 181, membrane 190 and retrieval wires 150, similar to the one described in FIGS. 7a-7d. The filter frame 181 is held in its collapsed state by surrounding sheath 130 in the first FIG. 10a.

In FIG. 10b the filter is delivered from sheath 130 by pushing sheath 130 more distally with manipulator 116 on main catheter 120, while filterholder tube 195 is held still at its manipulator 196.

FIG. 10c shows the step of delivery of the stent frame and valve in the annulus of the aortic valve, by withdrawing delivery sheath 175 over control wire 110 in proximal direction, while the filter is held in its axial position by means of holding manipulators 116 and 196 still. The sheath 175 is pulled all the way back until the stent frame is released completely, when sheath 175 approaches sheath 130.

Then, as shown in FIG. 10d, the control wire 110 is pulled back into sheath 175, through the delivered valve.

The filter can still stay open for some moments now, in order to enable the blood flow to flush all particles into the membrane. Finally, as shown in FIG. 10e, sheaths 175 and 130 are pulled more proximally, while manipulator 196 and filter holder tube 195 are held still. This results in a safe collapse of the filter mouth and the hole device can be removed from the patient's body.

If needed, the version described in FIGS. 8a-8e with extra tube 197 for allowing more radial and tangential freedom for the filter frame, may be used instead of the filter of FIGS. 7a-7d with only a single filter holder tube.

For a TAVI procedure the best location for placing the distal end of the filter frame is in the aortic arch, between the aortic valve and the brachiocephalic artery. The position of the perforated filter membrane itself is less relevant, because the filter frame holds the membrane entrance tightly against the aorta wall. So the membrane itself may either be located upstream of the entrance of the brachiocephalic artery, or cover it entirely or partly. In the latter case the perforations allow perfusion into the artery. Typical drilled circular perforations for protecting the brain against stroke are in the order of 100-120 microns in diameter, but other sizes and geometries, for example made by laser cutting, may be used as well. The hole geometry is also dependent on which filter is used. It may be a structure of fine metal or polymer wires made by any of well known techniques like braiding, crocheting, knitting, weaving or knotting, for example, and then the holes are not circular. Eventually the mesh structure may be dipped into a solution that contains glue, polyurethane or any other material, in order to secure the relative position of the fibers. This will prevent the undesired shape change of the perforations upon use. It is also possible that there are more than one filter frames, holding the filter membrane in place. The membrane can be attached to the frame in many ways, including suturing, laser bonding, laminating, embedding the frame struts between sandwich layers, welding, melting, gluing and so on.

The filter frame with attached fibers can also entirely be replaced by a metal frame such as Nitinol or the like. Instead of connecting the frame via flexible fibers to the catheter, the proximal and/or distal ends of the frame are directly connected to the catheter or filter holder tube, in order to enable delivery and retrieval. Of course, distinct frame sections need to have different functions. For example, the struts that run from the connection ring on the catheter to the section that supports the actual filter may have to be more flexible than the struts that are responsible for the radial expansion of the frame. Any filter design may be used in combination with a reverse sheath according to the present disclosure. Coatings on the contact surfaces can be used to reduce the friction.

Double Filter Frame Version

In all of the previous figures, only one filter frame is shown, but it is also possible to use a device with multiple frames.

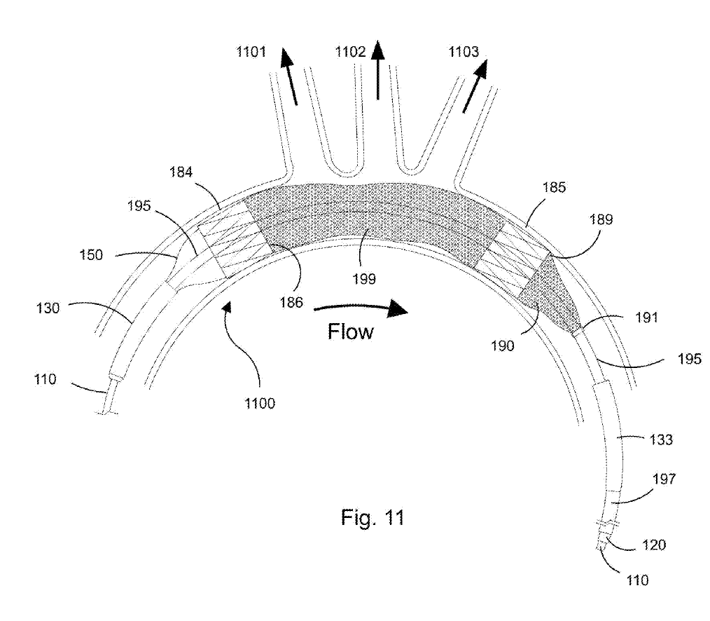

FIG. 11 shows a schematic view of the aorta arch 1100 with the three arteries above, from left to right the brachiocephalic 1101, left common carotid 1102 and left subclavian artery 1103. The blood flow is depicted with arrows. In this FIG. two filter frames 184 and 185 are shown. The distal frame 184 is located distally from the brachiocephalic artery 1101, while the proximal frame 185 is placed proximally from the left subclavian artery 1103.

Frames 184 and 185 may be frames similar to the ones described before, but now they are holding a relative long cylindrical perforated membrane 199 in between.

Frame 184 only holds the cylindrical membrane 199 open and firmly pressed to the inner aorta wall. It can be retrieved by pulling the main catheter 120 with the reverse sheath 130 over the frame by means of retrieval wires 150, as described before.

The second frame 185 is connected to the proximal end of cylindrical membrane 199. At the proximal side of the proximal frame 185 the apex of a conical membrane 190, like the one in FIGS. 9a-9e, is connected to the filter holder tube 195 with ring 191. Delivery and retrieval of the second filter frame 185 can be done by means of a regular proximal catheter sheath 133 on an extra catheter 197. Sheath 133 can be made long enough to enclose membrane 199, frame 185 and membrane 190, but it may also be shorter and only enclose frame 185 and membrane 190.

Such an elongated cylindrical membrane section 199 may be used to cover the entire upper aortic arch, including the entrances of the brachiocephalic artery, the left common carotid artery and the left subclavian artery. Membrane 199 actually does not capture debris, as it only prevents it to enter the three arteries. It acts as a deflector, guiding the debris to the conical filter 190, which is connected to the proximal side 189 of frame section 185 and which filters the remainder of the blood flow downstream the aorta.

Catheter 110 runs all the way from the proximal side until the distal end, where it is used for any procedure in the heart.

With a proper positioning of filter systems according to the disclosure it will give sufficient embolic protection for all arteries downstream. In this case the only arteries that do not have protection are the coronary arteries, because their entrances are located between the valve and landing zone for the filter in the aortic arch. The pore size of the conical membrane 190 may be identical as for the cylindrical membrane 199. However, for more protection against stroke the pore size of the cylindrical membrane 199 may be smaller than for the conical membrane 190.

The material of the reverse sheath may be relatively rigid, but can also be compliant. In the latter case it may work like the body of a snake that swallows a prey with a large size. As soon as the prey has passed the mouth of the snake, the outer diameter of the snake's head elastically returns to its normal size. If the entrance of the reverse sheath has a similar behavior, it becomes easier to get the filter frame into its collapsed state. Also, this entrance may have a small outside flare or taper to make withdrawal easier. It is also desirable to put marker bands on the reverse sheath and on the filter in order to be able to see the relative positions.

Filter with Non-Concentric Sheath

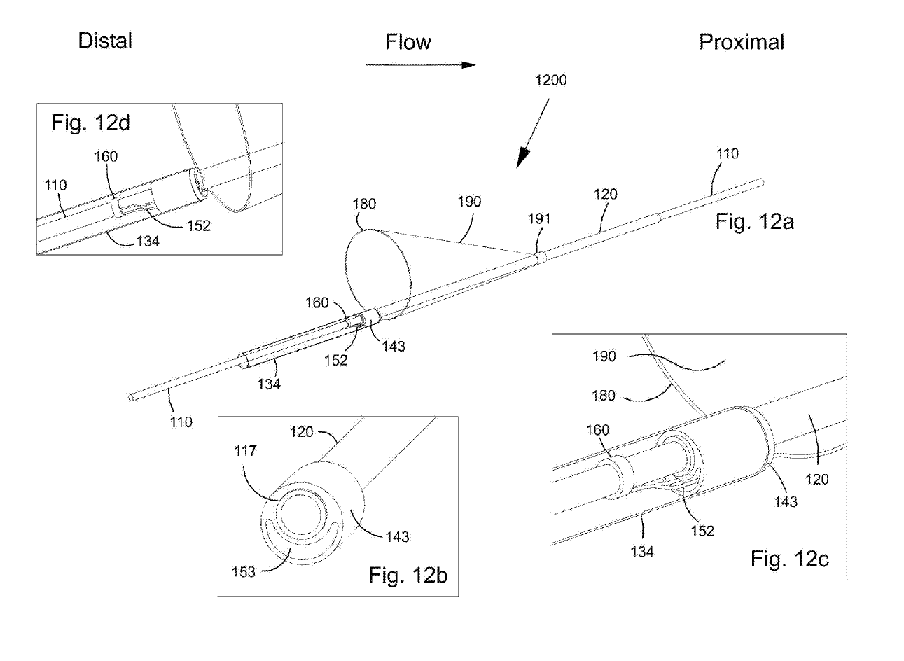

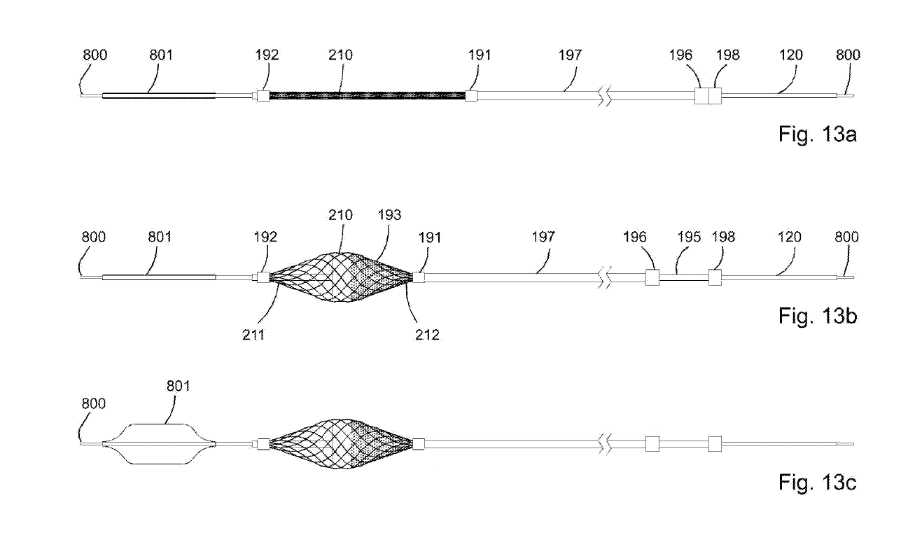

In the examples above the main catheter, the reverse sheath and the filter were always shown as concentric parts with a rotational symmetry, except for the example in FIG. 5. Other embodiments with non-concentric design are disclosed as well an example of which is given as a device 1200 in FIGS. 12a-d, where the main catheter 120 and reverse sheath 134 have a different length axis. The filter itself in this example is similar to the one shown in FIG. 5, with a self-expanding loop frame 180 with retrieval wires 152, a perforated membrane or mesh 190 attached thereto and a ring 191 at the apex, that fits on catheter 120.

FIG. 12a shows a control wire 110, main catheter 120 and a reverse sheath 134 connected to catheter 120 by means of ring 143. The distal end of catheter 120 is mounted in opening 117 of ring 143, as shown in detail in FIG. 12b. In ring 143 there is a separate second opening 153. FIGS. 12c and 12d give two detailed views from different angles for how the retrieval wires 152 are guided through the separate opening 153 to the control ring 160 that is mounted on control wire 110. When the main catheter and reverse sheath are pulled back over the control wire, the retrieval wires pull the loop 180 of the filter frame into the opening 153, thus collapsing the filter frame. The additional elongated sheath 134 holds the entire filter frame when it is collapsed, but after the loop frame has been closed, the membrane may stay outside of opening 152, at least for a part. Optionally the sheath 134 may be elongated in proximal direction in order to cover a larger part of the membrane when it is collapsed.

Filter without Reverse Sheath

The shapes of the filters, with or without any type of frames may be varied in numerous embodiments, but the examples given so far, with separate filter frame and retrieval wires, are just mentioned to show the function of the reverse sheath principle. Any combination of concentric or non-concentric devices having a main catheter plus reverse sheath for delivery and/or retrieval of any filter is meant to be part of the disclosure.

Besides the described embodiments with a reverse sheath, there is also an option to use many aspects of the examples above, but without a retrieval sheath. FIGS. 13a-c show an embodiment of a filter frame 210 that can be expanded by pushing its ends 211 and 212 towards each other. Contraction is achieved by enlarging the length of the frame 210. The frame itself may be made of a wire mesh or cut out of tubing, for example Nitinol with linear or superelastic properties. Filtration takes place by the mesh itself, or by an additional filter membrane 193, which is attached to the proximal side of the frame. This membrane is perforated or made of a fine woven material, for example. The distal end 211 of frame 210 is connected to the distal end of filter holder tube 195 by ring 192 and the proximal end 212 of frame 210 is connected to the distal and of outer tube 197 at ring 191. Outside the patient's body tubes 197 and 195 are each connected to manipulators, depicted as stops 198 and 196 respectively.

Several options are possible in such an embodiment. In the example of FIGS. 13a-c the frame 210 may be in its unbiased state while expanded. In that case manipulator stops 196 and 198 move automatically apart during spontaneous expansion of the filter, and have to be pulled closer together by force in order to close the filter. In this embodiment the filter frame 210 acts as a compliant self expanding system, which easily adapts to the present diameter and shape of the vessel. FIG. 13b gives the unbiased state and FIG. 13a gives the insertion state with stops 196 and 198 pushed together by force. FIGS. 13a-c further show an internal guidewire 800 and a balloon catheter 120 with a balloon 801 at the distal end.

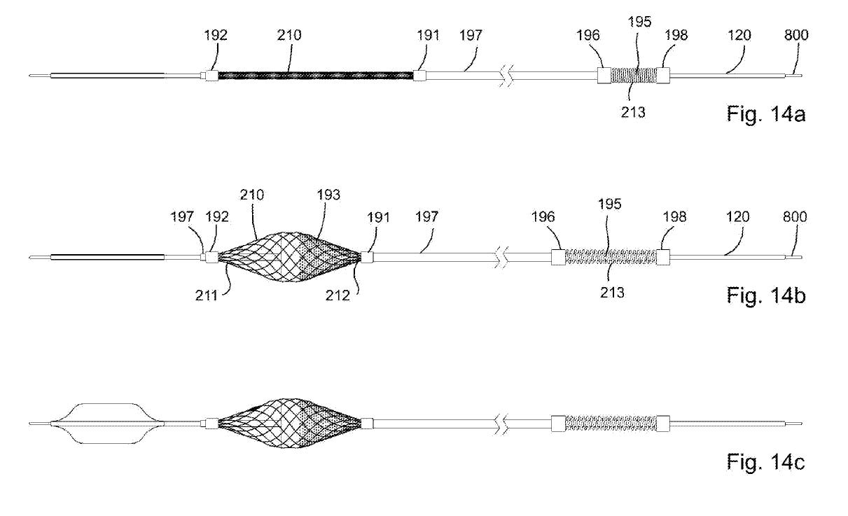

Alternatively the frame may be shape set in its stretched state, having its smallest delivery profile. FIGS. 14a-c show such an embodiment. In FIG. 14a frame 210 is shown in collapsed state, which is its unloaded rest position. Opening of the filter frame 210 is achieved by pushing the frame ends 211 and 212 together, by pushing manipulator rings or stops 196 and 198 on respectively outer tube 197 and filter holder tube 195 further apart. When stops 196 and 198 move closer again the frame 210 will collapse, entirely elastically or helped by actively moving stops 196 and 198 closer by external force by the operator. A compliant system can be achieved when an additional compression bias spring 213 is placed between the stops 196 and 198, where the spring actively tends to cause expansion of the frame. Such a compliant system automatically adapts its geometry upon variations in the radial compression forces on the expanded frame, which ensures a perfect wall apposition of the filter. Upon retrieval of the filter the operator has to push the bias spring to a smaller length, thus reducing the distance between stops and causing collapse of the frame. As an example, such as that depicted in FIGS. 13a-c, a balloon catheter is shown in combination with the filter device.

In the embodiments shown in FIGS. 13a-13c and 14a-14c, an optionally reverse sheath according to the disclosure may be used over the frame, or even a standard sheath from the proximal side. Such sheath versions have been described in the previous text and figures.

Example of a Prototype

Flexible fibers of UHMWPE (ultra-high molecular weight polyethylene), made by DSM in The Netherlands as Dyneema™, are extremely flexible and strong with a tensile strength of 20 times more than Nitinol. When the retrieval wires are made of 55 dTex fibers, the tensile strength is about 17 Newton. In the examples of prototypes according to FIGS. 7 and 10 the fibers are taken double, with a loop that is wrapped and secured by glue around connection anchors at the distal legs of the Nitinol frame 181 and connected to the control ring 192 on the filter holder tube 195 of FIGS. 7 and 10. The total cross section surface of such a double fiber bundle is only 1/900 mm², while 6 of these retrieval wires can pull together with 6×34=204 Newton, which is far more than needed to pull the frame into the sheath. A typical size for the delivery sheath of a TAVI catheter (such as that used for an aortic valve) is about 6 mm outer diameter and inner diameter 5.6 mm. The diameter of the main catheter is about 3.8 mm outer diameter. As the filter holder tube must slide easily over the main catheter, it has an inner diameter of 4 mm and outer diameter of 4.4 mm.

A TAVI catheter with a filter mounted on its surface should not have a larger profile than the existing catheter profile in order to prevent extra damage in the insertion site. If the reverse sheath profile has diameters of 6 mm OD by 5.6 mm ID it works well.

This means that the available space between the outer surface of the filter holder tube and inner surface of the reverse sheath is only 4.4 mm ID by 5.6 mm OD. In this gap, with a surface area of 9.4 mm², the filter holder tube, connection ring, retrieval wires and self expanding frame, as well as the filter membrane must fit. Therefor it is important to keep the dimensions of all components as small as possible, and the use of reinforcement fibers for the membrane is helpful in order to minimize the thickness of the membrane while the strength and reliability is maintained. Samples were made with a membrane of polyurethane skin, with reinforcement fibers of Dyneema™. The membrane thickness was varied for different samples by choosing the amount of steps during the dipping process of a PTFE cone in a solution of polyurethane in THF (tetrahydrofuran). A typical final thickness of about 20-30 microns for the membrane works well for reaching enough stability. The conical filter membrane was connected to a nitinol frame with 6 cells and expanded diameter of 30 mm. The filterframe was laser cut from tubing of 4 mm by 3.4 mm diameter and shape set on 30 mm.

The total cross section surface area for a 30 mm diameter membrane of 30 microns is only 2.8 mm² and for the frame it is 3.5 mm², so frame and membrane together fit easily in the gap between reverse sheath and filter holder tube. Providing the surfaces with a hydrophilic coating further helps reducing the friction in order to achieve a smooth delivery and withdrawal of the filter.

Instead of using a dipped and perforated membrane, a Dyneema™ fabric with a density of 25 gram/m² and a suitable pore size of about 100 microns can be used, because it is thin enough to fit in the reverse sheath together with the expandable frame. Another suitable mesh material may include a filter material made from fine-gauge nylon wires with well defined pore sizes.

Embodiments of the Disclosure

A reverse sheath may be used in any upstream procedure for opening up or widening of a more or less blocked artery, placing a stent or to prepare the annulus of a damaged heart valve for receiving a new valve. Such procedures include all types of diagnostic procedures or interventional treatments by means of a balloon, like drugs delivery from its surface or angioplasty, but also dissection methods for removing calcifications, stenosis, laser cutting, scoring balloons, lithotripsy systems and others. Balloon expandable stents or heart valve frames can be placed in combination with a filter with a reverse sheath according to the disclosure as well. One example is the TAVI-procedure where a heart valve is delivered. In particular, the procedure can be improved using the present filter as disclosed, by combining the delivery system for the valve with a filter as described on a single catheter. This saves costs, operation time, and reduces the risk as compared to using separate filtering devices.

As previously discussed, in one form, a central lumen may be made to extend all the way between the proximal and distal end of the system. In one form, a diagnostic or interventional treatment catheter may extend through the lumen. Likewise, in one form, the distal end of the treatment catheter may be placed at a location distally of the landing zone where the filter is placed. Moreover, the treatment may include procedures such as intravascular imaging, angioplasty, dissection, ablation, use of drug-eluting balloons, scoring balloons, laser cutting, drilling, reaming, dilating, lithoplasty, ultrasonic treatments, placement of stents, valves, stented valves, occluders, permanent filters and closure devices.

The balloon, filter and reverse sheath can be mounted on a common main catheter holding the same central control wire. Before inflation of the balloon the filter is pulled out of its delivery sheath. Optionally the filter is located inside the reverse sheath and a second filter delivery sheath and pulled out of both sheaths. One sheath may only contain the filter frame, while the other sheath contains the filter membrane. After deflation of the balloon the reverse sheath is pulled back, while the central control wire applies tension to the retrieval wires, finally causing the closure of the filter mouth. If needed, the remainder of the filter membrane may be collapsed by a second retrieval sheath, which is pushed forward over the first common catheter. However, full closure of the filter membrane may not be desirable and only closure of the filter mouth may be sufficient.

In another TAVI procedure a new heart valve in a self-expanding stent frame is placed inside the damaged original valve by means of a long catheter that is inserted through an incision in the patient's groin. One sheath delivers the valve, while a reverse sheath, coaxially located around the first catheter that holds the valve, delivers and retrieves the filter. Both sheaths can be moved independently. The filter is placed before the valve is deployed in order to capture any debris that may be dislodged during valve placement.

Besides balloon expandable TAVI-valves and self-expandable valves the disclosed filter can also be used in combination with other valve systems, such as those that have a frame that is mechanically expanded by axial contraction, followed by locking at the desired diameter. A catheter carrying a filter according to the present invention can be applied over the catheter that places this system.

The size and porosity of the filter membrane may be different for different types of procedures. In one embodiment the filter is only short and does not overlap with side arteries. It only filters the main stream in the aorta. In TAVI procedures the filter is preferably placed in the ascending aorta, between the valve annulus and brachiocephalic artery, in order to ensure protection against emboli entering the brains.

In another embodiment the filter is much longer and also covers the entrance of side arteries. In this embodiment the fine pores in the filter membrane allow the blood to enter into the side arteries, but emboli and other particles are deviated into the aorta and finally captured deeper into the filter.

In another embodiment the filter is connected to retrieval wires that run through side holes in the valve delivery sheath, these wires being connected to a ring that is mounted on a central control wire. During delivery of the valve the control wire is held in a fixed position, with the delivery sheath and stented valve located exactly in the area where the valve has to be seated. As soon as the valve delivery sheath is pulled back, the retrieval wires become stretched and the reverse sheath moves towards the filter opening. If delay is needed between valve delivery and filter closure, the device may be held in an intermediate position for a while, with the valve already in place.

Delivery of the valve is followed by closure of the filter in this way, using the same device, which can be removed from the patient then.

There may also be more than one expandable filter frame, mounted on the same catheter, for example when a long cylindrical filter membrane is located between two separate frame rings. In such cases the reverse sheath has to be long enough to be able to collapse both frames before retrieval, or retrieval can be done from two sides. First closing the distal filter mouth with the reverse sheath and finally closing the proximal frame with a conventional sheath, as will be described in the following examples.

In another embodiment a filter does not need a sheath for expansion or contraction. The filter catheter is placed on the catheter for treatment of the patient and the filter frame in expanded and collapsed by changing its length. The filter has its entrance for the blood is at the distal side and collects debris at the proximal side. Optionally a reverse sheath or a sheath from the proximal side may be used in combination with such a filter.

Filters according to the invention may be placed concentric or non-concentric on the catheter that is used for treatment in the patient's body.

For FIGS. 1 through 6a-6f and 12a-12d the “first tubular filter carrying catheter” in the claims is represented as the control wire 110 and the “second tubular catheter” is represented as main catheter 120, holding the sheath. In these figures the first catheter is longer than the second catheter, and protrudes at both sides of the second catheter, while the second catheter surrounds the first catheter.

For FIGS. 7a-7d and 11 the “first tubular filter carrying catheter” in the claims is represented as tube 195 and the “second tubular catheter” is represented as main catheter 120, holding the sheath. In these FIGS. the second catheter is longer than the first catheter, and protrudes at both sides of the first catheter, while the first catheter surrounds the second catheter.

For FIGS. 13a-13c and 14a-14c the “first tubular filter carrying catheter” in the claims is represented as tube 195 and the “third tubular catheter” is represented as catheter 197, its distal end connected to the proximal end of the filter frame. In these figures the second catheter is longer than the first catheter, and protrudes at both sides of the first catheter, while the first catheter surrounds the second catheter.

It is noted that defined terms like “reverse sheath”, “main catheter”, “delivery sheath”, “concentric”, “filter holder” and others are not utilized herein to limit the scope of the disclosed subject matter or to imply that certain features are critical, essential, or even important to the structure or function of the disclosed subject matter. Rather, these terms are merely intended to highlight alternative or additional features that may or may not be utilized in a particular embodiment of the present disclosure.

Likewise, for the purposes of describing and defining the present disclosure, it is noted that the terms “substantially” and “approximately” and their variants are utilized herein to represent the inherent degree of uncertainty that may be attributed to any quantitative comparison, value, measurement or other representation, as well as to represent the degree by which a quantitative representation may vary without resulting in a change in the basic function of the subject matter at issue.

While certain representative embodiments and details have been shown for purposes of illustrating the subject matter of the present disclosure, it will be apparent to those skilled in the art that various changes may be made without departing from the scope of the disclosure, which is defined in the appended claims.

While certain representative embodiments and details have been shown for purposes of illustrating the disclosure, it will be apparent to those skilled in the art that various changes may be made without departing from the scope of the disclosure, which is defined in the appended claims.

1. An embolic filtering system used to capture embolic debris in a body vessel with the blood flowing from the distal end towards the proximal end of the system, comprising:
an expandable and collapsible filter assembly including an expandable frame moveable between an expanded position and an unexpanded position, the expandable filter being disposed on a first tubular filter carrying catheter;
a flexible filtering element attached to and movable with the frame;
the unfiltered blood flow being able to enter the filtering assembly at a first distal entrance side;
the filtered blood leaving the filter assembly at a second proximal exit side;
the first filter carrying catheter having a proximal end outside the body and a distal section enclosed by the filter assembly with at least the distal end of the filter assembly attached thereto;
a second tubular catheter mounted coaxially with the first catheter, being movable in axial direction relative to the first catheter;
a reverse retrieval sheath connected to the distal end of the second catheter, the sheath having a distal end portion and a proximal end, the distal end portion being connected to said second catheter and the proximal end portion of the sheath having an opening, adapted to maintaining the filter assembly in the unexpanded position, deliver the expandable filter assembly for release and being axially movable to collapse and retrieve the filter assembly, starting the collapse at the distal entrance of the filter; and
manipulators, attached to the proximal ends of said first and second catheter to enable an operator to vary the relative axial position of both catheters.

2. The system of claim 1, comprising a third catheter disposed at the proximal side of the filter assembly and axially movable over the first catheter, the third catheter having a manipulator at its proximal end, and a distal end being adapted to pull the filter assembly out of the reverse sheath in order to allow the frame to expand upon delivery of the filter in a desired location in the vessel.

3. The system of claim 2, wherein the third catheter has a locally enlarged distal section which can contain at least a proximal part of the filter assembly in its unexpanded state during insertion or retrieval of the filtering system.

4. The system of claim 2, wherein the distal end of the third catheter is connected to the proximal end of the filter assembly, and wherein relative axial movement between the manipulators of said first and third catheter enable the operator to change the length of the filter frame.

5. The system of claim 4, wherein enlarging the length of the filter frame enables an easier retrieval of the filter into its reverse sheath.

6. The system of claim 4, wherein reducing the length of the filter frame causes a better wall apposition of the frame against the vessel wall.

7. The system of claim 4, wherein the expansion of the filter assembly is caused by a biasing spring that elastically changes the relative position of the manipulators of the first and third catheter.

8. The system of claim 7, wherein variations of radial compression forces on the frame are elastically compensated by the mechanical interaction between the frame and the biasing spring.

9. The system of claim 1, wherein the proximal end of the filtering assembly is also connected to the first filter carrying catheter.

10. The system of claim 1, wherein the first filter carrying catheter is located inside the second tubular catheter.

11. The system of claim 1, wherein the second tubular catheter is located inside the first filter carrying catheter.

12. The system of claim 1, wherein the frame is made from metals, polymers, laser cut tubing, woven, knitted or braided mesh structures including shape memory alloys like Nitinol with linear or superelastic properties.

13. The system of claim 1, wherein the flexible filtering element is made from a perforated polymer membrane, perforated sheet material, woven, braided or knitted mesh structures including shape memory alloys like Nitinol with linear or superelastic properties.

14. The system of claim 1, wherein the distal end of the frame is attached to the first filter carrying catheter with at least one retrieval wire.

15. The system of claim 14, wherein the retrieval wire is made of the same material as the frame.

16. The system of claim 14, wherein the retrieval wire is made of a different material as the frame.

17. The system of claim 1, wherein the distal end of the reverse retrieval sheath is connected to the distal end of the second catheter by a ring with at least one axial hole for guiding a retrieval wire to a point of attachment at the first catheter at a location distally from the sheath.

18. The system of claim 16, wherein the length axis of the reverse retrieval sheath is non-concentric with the length axis of the second catheter.

19. The system of claim 1, wherein an inflatable balloon is mounted on the outer surface of the reverse retrieval sheath, and wherein the proximal opening of the sheath gives access to said filter assembly.

20. The system of claim 19, wherein the outer surface of the reverse retrieval sheath is radially collapsible but resistant against buckling in length direction.

21. The system of claims 19-20, wherein the distal end of the reverse retrieval sheath has a perfusion lumen.

22. The system of claim 1, wherein said first catheter carries two filter frames, comprising:
a first filter frame at the first catheters distal end and a second filter frame more proximally;
the first frame adapted to be retrieved into the reverse sheath;
the second frame connected to a first flexible filtering element with pores that only allow particles below a critical size to flow through the main vessel in which the system is placed;
the second frame adapted to be retrieved into a proximal sheath;
a second flexible filtering element mounted between the proximal end of the first frame and the distal end of the second frame;
the first frame located distally from a side branch of said main vessel;
the second frame located proximally from the side branch; and
wherein the second flexible filtering element has pores that only allow particles below a critical size to flow into the side branch.

23. The system of claim 22, wherein the pore size of the first and second filtering elements is different.

24. The system of claims 1-23, comprising a central lumen running all the way between the proximal and distal end of the system, a diagnostic or interventional treatment catheter running through said lumen, and the distal end of the treatment catheter placed at a location distally of the landing zone where the filter is placed.

25. The system of claim 24, wherein the treatment comprises procedures including intravascular imaging, angioplasty, dissection, ablation, use of drug-eluting balloons, scoring balloons, laser cutting, drilling, reaming, dilating, lithoplasty, ultrasonic treatments, placement of stents, valves, stented valves, occluders, permanent filters and closure devices.

26. A method of using the embolic filtering system of claims 1-25.
